(19) United States
(12) Patent Application Publication
(10) Pub. No.: US 2003/0176470 A1
(43) Pub. Date: Sep. 18, 2003

(54) Topical composition containing at least one aryl oxime, and method for the preparation thereof

(76) Inventors: Joachim Bunger, Gross-Umstadt (DE); Jutta zur Lage, Darmstadt (DE); Michael Schwarz, Weiterstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/296,173

(22) PCT Filed: May 23, 2001
(86) PCT No.: PCT/EP01/05953

(30) Foreign Application Priority Data
May 24, 2000 (DE) 100 25 558.2

(51) Int. Cl. A61K 31/44; A61K 31/40; A61K 31/15; A61K 31/22

(52) U.S. Cl. 514/357; 514/408; 514/640; 514/546

(57) ABSTRACT

The present invention relates to a topical composition, comprising

(c) at least one aryl oxime of the Formula (I) and

(d) at least one emulsifier,

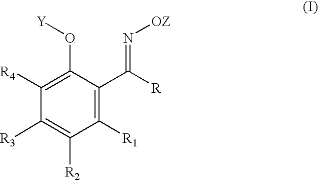

wherein:

Y, Z represent independently from each other H, C₁₋₁₈ alkyl, C₂₋₁₈ alkenyl, C₂₋₁₈ carboxy alkyl, C₃₋₁₈ carboxy alkenyl or C₂₋₁₈ alkanoyl;

R represents C₁₋₁₈ alkyl, C₂₋₁₈ alkenyl, C₃₋₈ cycloalkyl, aryl, aralkyl, heteroaryl, heteroaralkyl or condensed systems;

R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl,

wherein the component (b) is selected from the group consisting of an ester, the carboxylic acid residue of which is derived from C₅-C₁₆ acids and the hydroxyl residue of which is derived from monomers, dimers or trimers of lactic acid or one of its salts or a polyglycerin of 2 to 10 molecules of glycerin whereby 1 to 3 moles of carboxylic acid are present per mole of polyglycerin.

Furthermore, the present invention relates to a process for the preparation of said topical composition as well as the use of said topical composition.

The present invention relates to a topical composition, comprising at least one aryl oxime, said composition being stable over a prolonged period of time and ensuring good penetration into the skin. Furthermore, the present invention relates to a process for the preparation of said topical composition.

Inflammations are observed in many diseases as symptoms whereby these inflammations occur, either casually or as a secondary manifestation due to pathological changes. Moreover, they may be caused by external chemical or physical noxae. An inflammation is a multi-functional event of different morphological and functional factors. These factors concern in this connection disorders in the cellular area, in the blood circulation, inflammation-induced transudation and exudation, infiltration and proliferation. Together with these disorders further changes may occur, so that inter alia spongiosis, acanthosis or parakeratosis will occur.

During the release, course and the regulation of many of these events, mediating systems are involved. Thus, lymphokinines released from sensitized T-lymphocytes are substantially involved in the cellular immune response with a large number of biological effects (Schöpf, E., Korting, G. W. [Editor] Dermatologie u. Praxis Vol. 1, Thieme: Stuttgart, New York [1980]). Furthermore, in this connection, the effect of kinines, activated complementing factors, lysosomal enzymes, cyclic nucleotides and various epidermal factors are known. Prostaglandins and leukotrienes play a particular roll. As an example of a prostaglandin effect, a chemotactic effect on leukotrienes is known which decreases the vessel permeability chronologically after the kinines. Leukotrienes, on the other hand, act chemotactically on the granulocytes and influence the contractability and permeability of the vessels.

Apart from the histamine release, UV-B erythema is mediated by the arachidonic acid cascade, whereby an increased cyclo-oxygenase mediated prostaglandin synthesis, in. particular, of PGE₂ and PGF₂ occurs. The lipoxygenase pathway via 5-HPETE and LTA4 leads to the essential elements of the inflammation, such as cellular infiltration of the inflamed tissue and oedema formation (review in: Gallin, J., Goldstein, I. M., Snyderman, R., [Editor], Inflammation: Basic principles and clinical correlates, New York, Raven Press [1988]).

Different active agents for the treatment of inflammations are known. Corticosteroids have the greatest importance for the treatment of the mechanisms mentioned above, which lead to different skin diseases. Weak to medium strong corticosteroids, mainly non-fluorinated derivatives of hydrocortisone, are mainly employed for the therapy of inflammatory, allergic and pruriginous skin diseases. However, during a treatment with corticosteroids undesired side effects occur depending on the employed active agent, the type and duration of the treatment, whereby these side effects must be observed and taken into consideration by any means when using these substances (Review: Symposium in Topical Corticosteroids. In: Drugs Vol. 36, 5 [1988]). For these reasons, it is preferred to use non-steroidal anti-inflammatory active agents, whereby the therapeutic efficiency of the substances known to date is, however, very limited and in most cases below that of hydrocortisone. This concerns active agents such as salicylic acid, acetyl salicylic acid, bufexamac, bendazac, phenylbutazone, oxyphenbutazone, diflumidone, indometacine and, partially also, anti-histamines (Gloor, M., Pharmakologie dermatologischer Externa. Springer Verlag Berlin Heidelberg New York [1982]).

In EP-A-0149 242 1-(2-hydroxyaryl)-alkan-1-one oximes are suggested as active agents inter alia for the treatment of skin diseases. The active agents can be used in this context orally, perilingually, rectally, parentally, intravenously or percutaneously, as well as aerosols. As pharmaceutical formulations there are mentioned inter alia suspensions and emulsions, pastes, ointments, gels, creams and lotions. In an example 2-hydroxy-5-methyl-laurophenone oxime was applied intraperitonally and the inhibition of the carrageenine oedema of the foot of a rat was examined. :

Apart from the properties of the active agent and the morphological and functional condition of the skin areas to be treated, the therapeutic efficiency of the topically applied medicament is mainly dependent on the properties of the vehicle employed. In summary, a sufficient, optimum concentration-time profile of the active agent in the damaged skin layer is desired.

EP-B-389 773 discloses a process for the preparation of a galenic formulation with optimum bio-availability of the active agent 2-hydroxy-5-methyl-laurophenone oxime. In this case cholic acids are used as the resorbent. Cholic acids which can be used are, for example, deoxy or dehydrocholic acid or mixtures of these in the form of their salts. The formulation can be effected in the form of solutions, suspensions, capsules, granules, tablets or dragees.

In DE-A-41 16 123 there are disclosed topical compositions on the basis of 2-hydroxy-5-methyl-laurophenone oxime. As the areas of use, the pharmaceutical industry, human and veterinary medicine, as well as cosmetics are mentioned. Apart from the active agent, the formulations include furthermore hydrating substances typically present in the skin and/or moisturizer and/or penetration promoters and other compounds. The concentration of the active agent is typically 0.1 to 50%, and the additional substances mentioned above are present in an amount of 0.1 to 40%. As a preferred hydrating substance and/or moisturizer urea is used. Preferred penetration promoters include propylene glycol and cholic acid. The topical formulation is present in the form of W/O-emulsions, hydrogels or mixed gels in the form of water-free and/or water-containing and lipophilic ointments, water-containing lipophilic ointments or non-ionic creams, pastes, shaking mixtures, lotions and emulsions. The incorporation of urea can increase the penetration of the active agent into the different layers of the human skin as well as the liberation of the active agent from the topical formulation. The addition of propylene glycol and sodium deoxy cholate as penetration promoters can further improve this effect.

A major problem with the topical use of the compounds mentioned above is their low solubility in cosmetic and dermatological formulations and their tendency to crystallize out of the formulation. In particular, the active agent is soluble in the emulsion systems by heating, but the formation of crystals is observed both in OMw as well as in W/O emulsions during storage. Even if the active agent remains soluble within the emulsion systems, the formation of crystals in the skin layers may occur after application of the formulation.

Numerous studies were conducted in order to obtain optimum compositions of the active agent in which an improvement of solubility and, a prevention of the formation of crystals is achieved. Although aryl oximes, in particular, 2-hydroxy-5-methyl-laurophenone oxime, have a good solubility in different solvents such as isopropanol, acetone, chloroform, 2-phenylethanol and Triton-X100, these carriers are not, or only limited, suitable for use in cosmetic and dermatological formulations. Therefore, the carriers must be non-toxic, non-carcinogenic and skin compatible and, furthermore, should be odour neutral. Moreover, these carriers, e.g. solvents or emulsifiers, should be compatible with the major part of the topical composition, i.e. an aqueous phase or an oil phase, and should not form a separate phase or lead to the precipitation of the active agent.

During investigations regarding the solubility of, in particular, 2-hydroxy-5-methyl-laurophenone oxime, solutions could be prepared which are stable after 4 days at room temperature under the exclusion of UV light. These solutions were ethanolic solutions of the active agent which included further components such as 0.8/1.0 and 2.0% PEG600 or PEG6000. The active agent was present in these solutions in an amount of 10 wt. %. When the amount of the active agent was increased, the formation of crystals was observed. These solutions can be incorporated into water in order to prepare topical formulations. When the amount of the solution of the active agent in the formulation was 10 to 20 wt. %, however, in some cases the formation of crystals of 2-hydroxy-5-methyl-laurophenone oxime occurred.

Furthermore, typically an unwanted discoloration, e.g. a yellow discoloration, occurs when the active agent is brought into one of the formulations mentioned above.

It is, therefore, the object of the present invention to provide topical compositions comprising at least one aryl oxime, which show an improved solubility, as well as a stabilization of the active agent, and which prevent the formation of crystals of the active agent, so that an increase of the bio-availability of the aryl oxime is achieved. In addition, the composition should ensure that discolorations are prevented, a good penetration to the desired site of action, a good liberation of the active agent, and a good skin compatibility is achieved.

This object is achieved with a topical composition, comprising:

(a) at least one aryl oxime of Formula (I) and

(b) at least one emulsifier

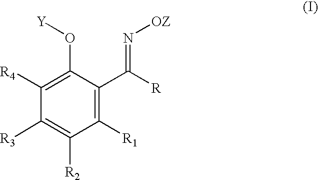

wherein,

Y, Z represent independently from each other H, C1-18 alkyl, C₂₋₁₈ alkenyl, C₂₋₁₈ carboxy alkyl, C₃₋₁₈ carboxy alkenyl or C₂₋₁₈ alkanoyl;

R represents C₁₋₁₈ alkyl, C₂₋₁₈ alkenyl, C₃₋₈ cycloalkyl, aryl, aralkyl, heteroaryl, heteroaralkyl or condensed systems;

R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl,

wherein component (b) is selected from the group consisting of at least one ester, the carboxylic acid residue of which is derived from C₅-C₁₆ acids and the hydroxyl residue of which is derived from monomers, dimers or trimers of lactic acid or one of its salts or a polyglycerin of 2 to 10 molecules of glycerin whereby 1 to 3 moles of carboxylic acid are present per mole of polyglycerin.

The present invention also relates to a process for the preparation of the aforementioned topical composition, comprising the steps

preparation of a phase A by mixing the components (a) and (b), and

incorporation of the obtained phase A into phase B containing a carrier.

The topical composition in accordance with the present invention is suitable for the prophylaxis and/or treatment of skin diseases and/or inflammation responses of the skin. Furthermore, the topical composition in accordance with the present invention can be used for the cosmetic care of the skin.

Surprisingly, it was found that the aryl oximes are prevented from being crystallized out, when they are present together with certain emulsifiers, which integrate these compounds into a liquid-crystalline (LC) phase and which can stabilize them in this state. In this case, a so-called liposomal encapsulation occurs which can permanently suppress any crystallization phenomena. Essentially the compatibility between the structure of the active agent to be encapsulated and the respective carrier system is decisive for the stabilization of these vesicle shaped membranes. In this case the complete solubility in the vesicle forming emulsifier is a requirement. The stabilization also prevents a discoloration of the composition.

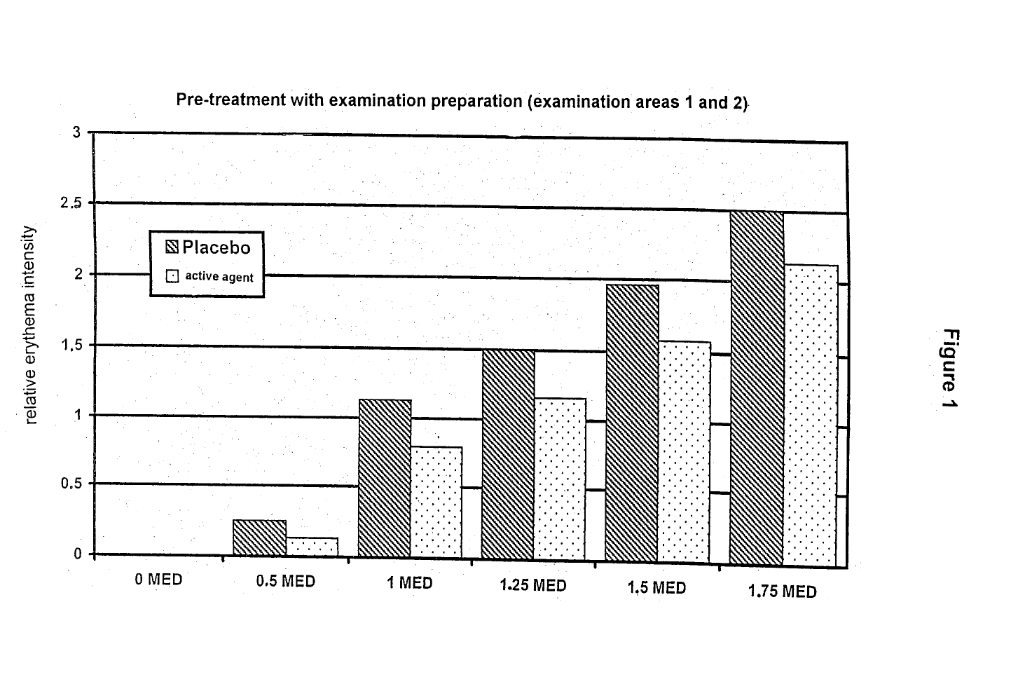

FIG. 1 shows the results of the visual assessment conducted in example 7 for the determination of the intensity of the erythema of the examination areas 1 and 2 in which a pre-treatment with the respective examination preparations took place.

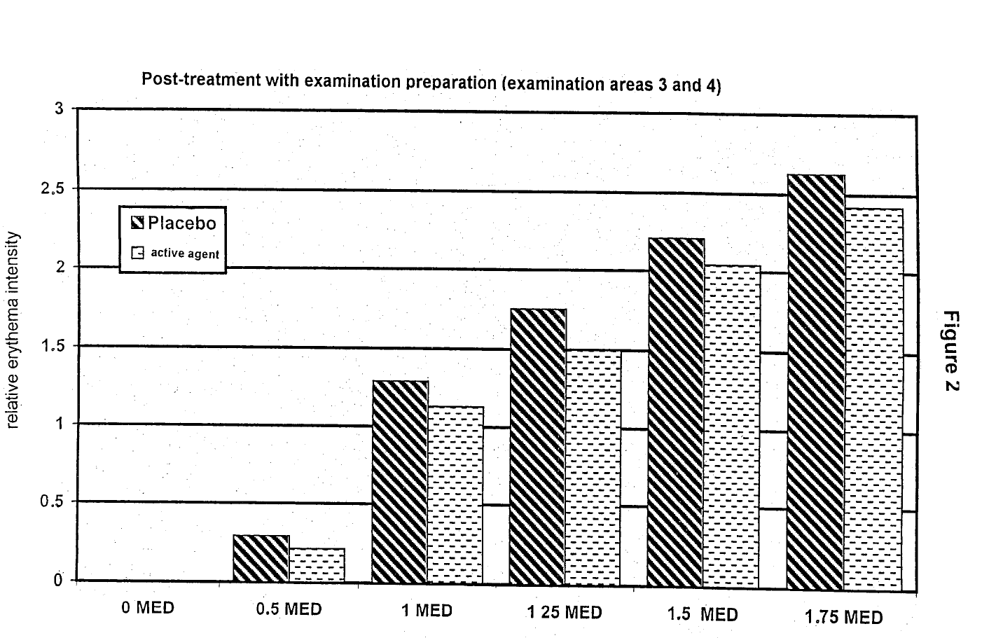

FIG. 2 shows the results of the visual assessment conducted in example 7 for the determination of the intensity of the erythema of the examination areas 3 and 4 in which a post-treatment with the respective examination preparations took place.

The topical composition in accordance with the present invention comprises as component (a) at least one aryl oxime of Formula (I).

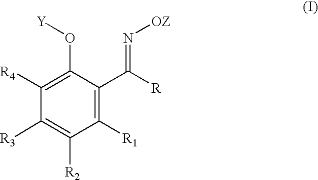

wherein,

Y,Z represent independently from each other H, C₁₋₁₈ alkyl, C₂₋₁₈ alkenyl, C₂₋₁₈ carboxy alkyl, C₃₋₁₈ carboxy alkenyl or C₂₋₁₈ alkanoyl;

R represents C₁₋₁₈ alkyl, C₂₋₁₈ alkenyl, C₃₋₈ cycloalkyl, aryl, aralkyl, heteroaryl, heteroaralkyl or condensed systems;

R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl.

Alkyl, alkenyl, carboxy alkyl, carboxy alkenyl, alkanoyl, cycloalkyl, alkoxy, aryl, aryloxy, and aralkyl can be unsubstituted or substituted. Suitable substituents of these groups are preferably alkyl, alkoxy, alkenyl, aryl, aryloxy, aralkyl, heteroaryl, heteroaralkyl, hydroxy, carboxy, carboxy alkyl, dialkyl amine, sulfonyl and combinations thereof.

Alkyl is respectively straight chain or branched alkyl and is therefore preferably methyl, ethyl, propyl, isopropyl, butyl, isobutyl, t-butyl, pentyl, hexyl, heptyl, octyl, nonyl, decyl, undecyl, dodecyl, tridecyl, tetradecyl, pentadecyl, hexadecyl, heptadecyl and octadecyl.;

Alkenyl means that in the specified alkylene moiety one or more double bonds may be present.

Aryl is an aromatic C₆₋₂₀ hydrocarbon residue and is prefereably phenyl.

Aralkyl is an alkyl group substituted with aryl and has preferably the meaning of benzyl or phenethyl.

Cycloalkyl is a cyclic alkyl group and is preferably cyclopropyl, cyclobutyl, cyclopentyl, cyclohexyl, cycloheptyl or cyclooctyl.

Heteroaryl is an aromatic ring with heteroatoms, preferably a nitrogen-containing ring, such as pyridyl or pyrimidyl.

Heteroaralkyl means a alkyl group substituted with heteroaryl and is preferably pyridyl methyl and pyrimidyl methyl.

Suitable condensed systems are preferably the residues naphthyl, benzofuryl, quinolinyl, indolyl or cinnolinyl.

Dialkyl amine means NR₅R₆ wherein R₅ and R₆ may be the same or different, and C₁₋₁₂ alkyl.

Z and Y are preferably independently from each other a hydrogen atom, a C₁₋₆ alkyl group which can have at least one substituent selected from —OH, —COOH, —SO₃H or NR₅R₆, an alkanoyl group represented by —C(O)R₇ wherein R₇ is a C₁₋₆ alkyl group which may have at least one substituent selected from —OH, —COOH or —SO₃H, or a CONHR₈ group wherein R₈ is a C₆₋₂₀ aryl group. Particulary preferably Z and Y are independently from each other a hydrogen atom, —(CH₂)₁₋₆COOH, —CH₂CH(OH)CH₂OH, —(CH₂)₁₋₆SO₃H, —(CH₂)₁₋₆NR₅R₆ or C(O)(CH₂)₁₋₆COOH.

Substituent R is preferably a C₁₋₁₂ alkyl group, particularly preferred are C₁₋₅ and C₁₁ alkyl groups.

Substituent R₁ is preferably a hydrogen atom or a chlorine atom.

Substituent R₂ is preferably a hydrogen atom or a chlorine atom or a C₁₋₆ alkyl group. Particularly preferred are a hydrogen atom, a chlorine atom and a methyl group.

Substituent R₃ is preferably a hydrogen atom or a C₁₋₆ alkyl group, a C₁₋₆ alkoxy group, a O-cyclohexyl group or a benzyl group.

Substituent R₄ is preferably a hydrogen atom or a chlorine atom.

R₁, R₂, R₃ and R₄ may be preferably substituted, if possible, with —OH, —COOH, —SO₃H or —NR₅R₆ to increase, e.g. the water solubility.

Preferred examples of component (a) include:

4-methyl-2-hydroxy-caprophenone oxime, 5-methyl-2-hydroxy-caprophenone oxime, 5-methyl-2-hydroxy-caprophenone (N-phenylcarbamoyl) oxime, 5-methyl-2-hydroxy-laurophenone oxime (2-hydroxy-5-methyl-laurophenone oxime), 3-chloro-2-hydroxy-caprophenone oxime, 4-pentoxy-2-hydroxy-acetophenone oxime, 4-decyloxy-2-hydroxy-acetophenone oxime, 4-benzyloxy-2-hydroxy-acetophenone oxime, 4-decyloxy-2-hydroxy-propiophenone oxime, 4-butoxy-5-n-hexyl-2-hydoxy-acetophenone oxime, 4-pentoxy -2-hydroxy-caprophenone oxime, 4-decyloxy-2-hydroxy-caprophenone oxime, 4-octyloxy -2-hydroxy-laurophenone oxime, 4-cyclohexyl-oxy-2-hydroxy-propiophenone oxime, 5-chloro-2-hydroxy-caprophenone oxime, 3-chloro-2-hydroxy-laurophenone oxime, 5-chloro -2-hydroxy-laurophenone oxime, 4-butoxy-2-hydroxy-acetophenone oxime, 4-dodecyloxy -2-hydroxy-propiophenone oxime, 4-hexadecyloxy-2-hdroxy-acetophenone oxime, 4-octadecyloxy-2-hydroxy-acetophenone oxime, 4-decyloxy-2-hydroxy-laurophenone oxime, as well as the following oxime derivatives of 2-hydroxy-5-methyl-laurophenone oxime:

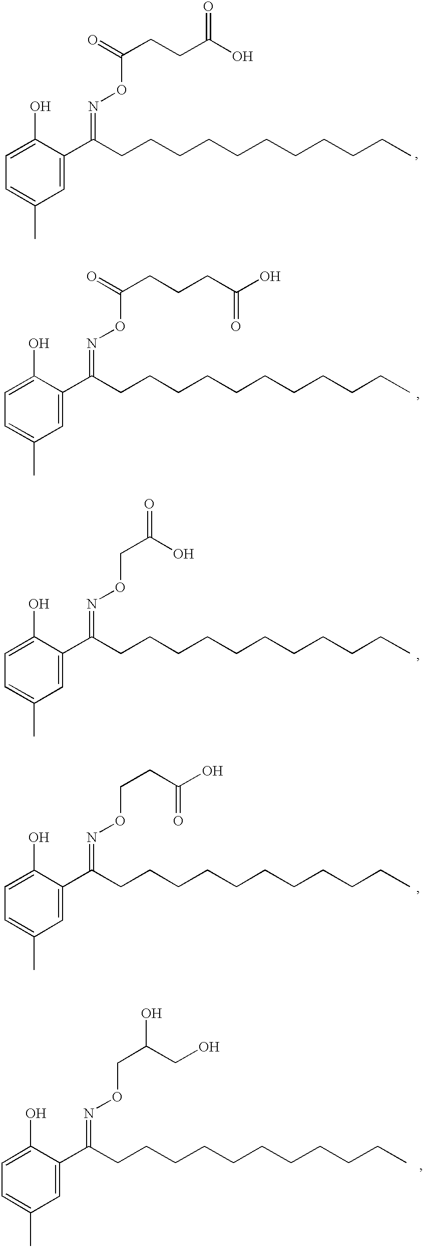

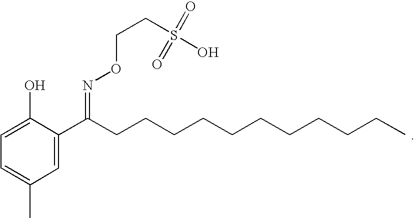

Particularly preferred are 2-hydroxy-5-methyl laurophenone oxime as well as its oxime derivatives mentioned before.

Component (a) is present in the composition according to the present invention in a sufficient amount to be suitable for a cosmetic or dermatological use. Typically, component (a) is present in the composition of the present invention in an amount of 0.05 to 5 wt %, preferably 0.02 to 2 wt %, more preferably 0.05 to 1.5 wt %.

The composition of the present invention comprises as component (b), at least one emulsifier, which is selected from the particular esters as mentioned above. The carboxylic acid residue of these esters is derived from the C₅-C₁₆ acids, preferably C₈₋₁₂ acids. The carbon chain of the carboxylic acid residue can be saturated or partially unsaturated. Preferred examples of the carboxylic acid residue include hexanoic acid, caprylic acid, capric acid, lauric acid, myristic acid, palmitic acid, stearic acid, behenic acid, oleic acid, linoleic acid and mixtures thereof, for example coconut fatty acid (the carboxylic acid residues of which are characterised by “cocoyl”) which is a mixture of the aforementioned fatty acids.

The hydroxy residue of the ester can be derived from monomers, dimers or trimers of lactic acid or one of its salts. Preferably a monomer or dimer of lactic acid is employed. It is furthermore preferred that the lactic acid is used in the form of its salt, i.e. as the lactate. Particularly preferred are alkali metal and alkaline earth metal salts, whereby sodium salts are particularly mentioned. Moreover, the hydroxy residue of the ester can be derived from a polyglycerin of 2 to 10 molecules of glycerin. In this case 1 to 3 moles of carboxylic acid are present per mole of polyglycerin. Particularly preferred 2 to 3 moles of carboxylic acid are present per mole of polyglycerin.

Examples of component (b) which is present in the composition of the present invention include dispersing auxiliaries as mentioned in DE-A-197 22 405, column 2, lines 38 to 56 as well as in the examples. Preferred are polyglycerin 10-tricaprylate, polyglycerin 10-trilaurate, polyglycerin 2-oleate, sodium lauryl lactate, sodium cocoyl lactate, capric/caprylic acid triglyceride and mixtures thereof. Particularly preferred are polyglycerin 2-oleate and sodium cocoyl lactate.

Component (b) is present in the topical composition in accordance with the present invention in a sufficient amount in order to integrate the active agent (component (a)) into a liquid-crystalline (LC) phase and in order to stabilize same in this state. Typically component (b) is contained in the topical composition of the present invention in an amount of 0.5 to 30 wt. %, preferably 0.5 to 20 wt. %, more preferably 1 to 10 wt. %.

In order to further improve the stability of the topical composition of the present invention with regard to an improved solubility and a prevention of the formation of the crystals of the active agent (component (a)) and in order to simultaneously ensure the stability of the topical composition of the present invention as such, the topical composition of the present invention contains, preferably furthermore, as component (c) at least one co-emulsifier selected from glycerin and sorbitan ester derivatives as well as cetearyl alcohol and ester derivatives thereof and mixtures of these substances. These co-emulsifiers do no have a disrupting effect on the construction of liquid crystalline gel networks which should form the basic structure of the topical composition in accordance with the present invention. The glycerin, sorbitan and cetearyl ester derivatives are typically derived from esters whereby the carboxylic acid residues of which are derived from C₅₋₁₆ acids, the carbon chains of which are saturated or partially unsaturated. Particularly preferred of these are glycerin stearate, sorbitan stearate, sorbitan isostearate, sorbitan diisostearate, sorbitan dioleate, sorbitan distearate, sorbitan laurate, sorbitan palmitate, sorbitan sesquiisostearate, sorbitan sesquioleate, sorbitan triisostearate, sorbitan trioleate, sorbitan tristearate, cetearyl octanoate, cetearyl palmitate, cetearyl isononanoate and mixtures thereof.

Component (c) is present in the topical composition in accordance with the present invention in a suitable amount in order to further stabilize the topical composition. Typically, the component (c) is contained in the topical composition of the present invention in an amount of 0.1 to 40 wt. %, preferably 0.5 to 15 wt. %, more preferably 1 to 10 wt. %.

It is preferred that, in addition, at least one lipophilic solvent as component (d) is present in order to improve the solubility of the active agent in the composition of the present invention. Typical lipophilic solvents suitable for a topic formulation include dimethicone cyclomethicone, mineral oil, isostearyl isostearate, octyl palmitate, propylene glycol/dicaprate/dicaprylate, C₁₂₋₁₅ alkyl benzoate, octyl decanol, ether derivatives of cetyl alcohol such as Ceteth-1, Ceteth-2, Ceteth-3,Ceteth-4, Ceteth-5, Ceteth-6 and Ceteth-10, ethylbutylacetyl aminopropionate, ethanol, isopropanol, isopropyl myristate, and mixtures thereof. Of these ethylbutyl acetyl aminopropionate, ethanol, isopropanol, isopropyl myristate and mixtures thereof are particularly preferred. The addition of the lipophilic solvent can improve the solubility of the active agent so that the content of the active agent (component (a)) within the composition of the present invention can be increased. When adding component (d), the amount of component (a) in the composition of the present invention is preferably in the range of 0.01 to 30 wt %.

Component (d) is typically present in the composition of the present invention, in a sufficient amount in order to improve the solubility of the active agents, and it is preferably present in an amount of 0.1 to 20 wt %, more preferably 0.3 to 17 wt %.

Furthermore, it is preferred that as a component (e), at least one auxiliary, selected from antioxidants and UV filters, is contained in the composition of the present invention.

Apart from the known effects of the antioxidants and UV filters such as the protection from cell damage by radicals, or the protection from UV radiation and its damaging effects, the antioxidants and/or UV filters can further stabilize the active agents (component (a)). This has the effect of, e.g. an advantageous increase of the storage stability of the composition of the present invention.

The following antioxidants known from references in the respective field can be present in the compositions of the present invention, for example flavonides, coumaranones, amino acids (e.g. glycine, histidine, tyrosine, tryptophan) and derivatives thereof, imidazoles (e.g. urocanic acid) and derivatives thereof, peptides such as D,L-camosine, D-camosine, L-carnosine and derivatives thereof (e.g. anserine), carotenoides, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthio uracil and other thiols (e.g. thioredoxin, glutathione, cysteine, cystine, cystamine as well as glycosyl, N-acetyl, methyl, ethyl, propyl, amyl, butyl, and lauryl, palmitoyl, oleyl, γ-linoleyl, cholesteryl and glyceryl esters of these) as well as salts of these, diaurylthiodipropionate, distearylthiodipropionate, thiodipropinoic acid and derivatives (esters, ethers, peptides, lipids, nucleotides, nucleosides and salts) of these as well as sulfoximine compounds (e.g. buthionine sulfoximines, homocysteine sulfoximine, buthionine sulfones, penta-, hexa-, heptathionine sulfoximine), furthermore (metal) chelating agents (e.g. α-hydroxy fatty acids, palmitic acid, phytic acid, lactoferrin), α-hydroxy acids (e.g. citric acid, lactic acid, malic acid), humic acid, cholic acid, cholic extracts, bilirubin, biliverdin, EDTA, EGTA and derivatives thereof, unsaturated fatty acids and derivatives thereof, vitamin C and derivatives (e.g. ascorbyl palmitate, magnesium ascorbyl phosphate, ascorbyl acetate) as well as coniferyl benzoate of the benzoic resin, rutinic acid and derivatives thereof, α-glycosyl rutin, ferulic acid, furfurylidene glucitol, carnosine, butylhydroxy toluene (BHT), butylhydroxy anisole, nordohydroguaiaretic acid, trihydroxybutyrophenon, uric acid and derivatives thereof, mannose and derivatives thereof, zinc and derivatives e.g. ZnO, ZnSO₄), selenium and derivatives thereof (e.g. selenium methionine), stilbenes and derivatives thereof (e.g. stilbene oxide, trans-stilbene oxide) and melatonin.

Mixtures of antioxidants are also suitable for use in the compositions of the present invention. Known and commercially available mixtures are, for example, mixtures containing, as active ingredients lecithin, L-(+)-ascorbyl palmitate and citric acid (e.g. Oxynex® AP), natural tocopherols, L-(+)-ascorbyl palmitate, L-(+)-ascorbic acid and citric acid (e.g. Oxynex® K LIQUID), tocopherol extracts from natural sources, L-(+)-ascorbyl palmitate, L-(+)-ascorbic acid and citric acid (e.g. Oxynex® L LIQUID), DL-α-tocopherol, L-(+)-ascorbyl palmitate, citric acid and lecithin (e.g. Oxynex® LM) or butylhydroxy toluene (BHT), L-(+)-ascorbyl palmitate and citric acid (e.g. Oxynex® 2004).

In a preferred embodiment of the invention the composition of the present invention contains as the antioxidant butylhydroxy toluene.

In another preferred embodiment the topical composition of the present invention contains as the antioxidant one or more compounds, selected from flavonoides and/or coumaranones.

Flavonoides are understood as being the glycosides of flavanones, flavones, 3-hydroxyflavones (=flavanoles), aurones, isoflavones and rotenoides (Rompp Chemie Lexikon, Vol. 9, 1993). Within the scope of the present invention, however, also the aglycones, i.e. the sugar free components, and the derivatives of the flavonoides and the aglycones are embraced. Within the scope of the present invention coumaranones embrace also the derivatives thereof.

Preferred flavonoides are derived from flavanones, flavones, 3-hydroxyflavones, aurones and isoflavones, in particular flavanones, flavones, 3-hydroxyflavones and aurones.

Flavanones are characterised by the following basic structure:

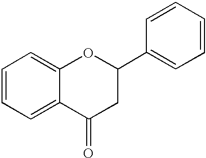

Flavones are characterised by the following basic structure:

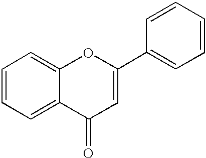

3-Hydroxyflavones (flavanoles) are characterised by the following basic structure:

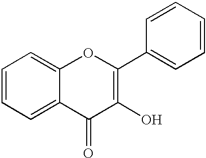

Isflavones are characterised by the following basic structure:

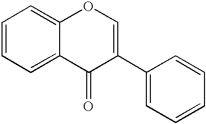

Aurones are characterised by the following basic structure:

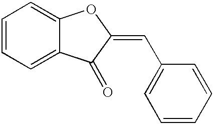

Coumaranones are characterised by the following basic structure:

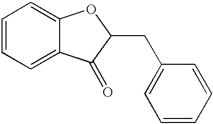

Preferably the flavonoides and coumaranones are selected from compounds of Formula (1):

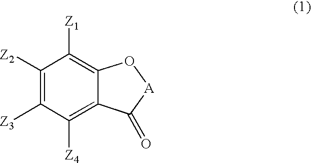

wherein

Z₁ to Z₄ each represent independently from each other H, OH, alkoxy, hydroxyalkoxy, mono- or oligoglycoside residues, whereby the alkoxy and hydroxyalkoxy groups can be branched or unbranched and may have 1 to 18 C-atoms and wherein on the hydroxy groups of the above mentioned residues, sulphate or phosphate may also be bonded,

A is selected from the group consisting of the substructures (1A), (1B) and (1C)

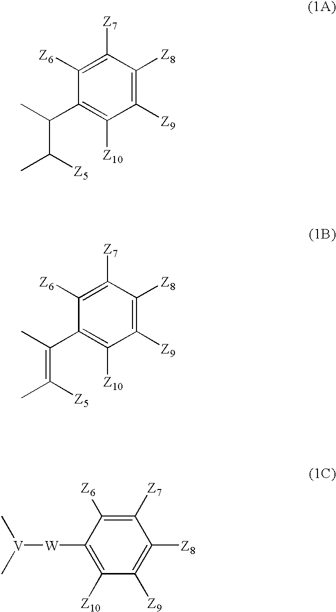

Z₅ represents H, OH or OR,

R represents a mono- or oligoglycoside residue,

Z₆ to Z₁₀ have the meaning of the residues Z₁ to Z₄ and

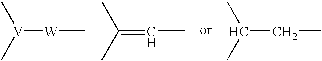

The alkoxy groups are preferably linear and have 1 to 12, preferably 1 to 8 C-atoms. These groups correspond, therefore, to Formula —O—(CH₂)ₘ—H, wherein m represents 1,2,3,4,5,6,7 or 8 and in particular 1 to 5.

The hydroxyalkoxy groups are preferably linear and have 2 to 12, preferably 2 to 8 C-atoms. These groups represent, therefore, Formula —O—(CH₂)ₙ—OH wherein n represents 2,3,4,5,6,7 or 8, preferably 2 to 5 and in particular preferably 2.

The mono- and oligoglycoside residues are preferably made up from 1 to 3 glycoside units. Preferably these units are selected from the group of hexosyl residues, in particular rhamnosyl residues and glycosyl residues. However, other hexosyl residues, for example allosyl, altrosyl, galatosyl, gulosyl, idosyl, mannosyl and talosyl can be used advantageously under the circumstances. Moreover, it can be advantageous for the invention to use pentosyl residues.

In a preferred embodiment the substituents have the following meaning

Z₁ and Z₃ the meaning H,

Z₂ and Z₄ a different meaning than H, in particular they represent OH, methoxy, ethoxy or 2-hydroxyethoxy,

Z₅ the meaning H, OH or a glycoside residue made up of 1 to 3, preferably 1 or 2 glycoside units.

Z₆, Z₉ and Z₁₀ represent the meaning H, and

Z₇ and Z₈ a different meaning than H, in particular they represent OH, methoxy, ethoxy or 2-hydroxyethoxy.

In another preferred embodiment, in particular when the water solubility of the flavonides and coumaranones should be increased, a sulphate or phosphate group is bonded to the hydroxy groups. Suitable counter-ions are, for example, ions of alkali metals or alkaline earth metals, wherein these are, e.g. selected from sodium or potassium.

In another preferred embodiment the flavonoides are selected from the following compounds: 4,6,3′, 4′-tetrahydroxyaurone, quercetin, rutin, isoquercetin, anthocyanidin (cyanidin), eriodictyol, taxifolin, luteolin, trishydroxyethylquercetin (troxequercetin), trishydroxyethylrutin (troxerutin), trishydroxyethylisoquercetin (troxeisoquercetin), trishydroxyethylluteolin (troxeluteolin) as well as sulphates and phosphates thereof.

From the flavonoides, rutin and troxerutin are particularly preferred. Especially preferred is troxerutin.

From the coumaranones, 4,6,3′, 4′-tetrahydroxybenzyl coumaranone-3 is preferred.

The antioxidants are typically incorporated into the compositions of the present invention in an amount of 0.001 to 5 wt %, preferably 0.5 to 5 wt %.

Suitable organic UV filters can be selected from all known UVA but also UVB filters known to the person skilled in the art. For both UV ranges there are reliable substances known from special literature, for example

Benzylidene camphor derivatives, such as

3-(4′-methylbenzylidene) dl-camphor (e.g. Eusolex® 6300),

3-benzylidene camphor (e.g. Mexoryl® SD),

polymers of N-{(2 and 4)-[(2-oxobom-3-ylidene)methyl]benzyl} acrylamide (e.g. Mexoryl® SW)

N,N,N-trimethyl-4-(2-oxoborn-3-ylidene methyl)anilinium methylsulphate (e.g. Mexoryl® SK) or

α-(2-oxoborn-3-ylidene)toluene 4-sulphonic acid (e.g. Mexoryl® SL).

Benzoyl or dibenzoyl methanes, such as

1 -(4-tert-butylphenyl)-3-(4-methoxyphenyl) propane 1 ,3-dione (e.g. Eusolex® 9020) or

4-isopropyldibenzoyl methane (e.g. Eusolex® 8020)

Benzophenones, such as

2-hydroxy-4-methoxy benzophenone (e.g. Eusolex® 4360) or

2-hydroxy-4-methoxy benzophenone 5-sulphonic acid and its sodium salt (e.g. Uvinul® MS-40).

Methoxy cinnamates, such as

p-methoxy cinnamic acid 2-ethyihexylester (e.g. Eusolex® 2292),

p-methoxy cinnamic acid isopentylester, e.g. as a mixture of the isomers (e.g. Neo Heliopan® E 1000),

Salicylate derivatives, such as

2-ethylhexyl salicylate (e.g. Eusolex® OS),

4-isopropylbenzyl salicylate (e.g. Megasol®) or

3,3,5-trimethylcyclohexyl salicylate (e.g. Eusolex® HMS),

4-aminobenzoic acid and derivatives thereof, such as

4-aminobenzoic acid,

4-(dimethylamino)benzoic acid 2-ethylhexylester (e.g. Eusolex® 6007),

ethoxylated 4-aminobenzoic acid ethylester (e.g. Uvinul® P25),

and further substances such as

2-cyano-3,3-diphenyl acrylic acid 2-ethylhexylester (e.g. Eusolex® OCR),

2-phenylbenzimidazole 5-sulphonic acid as well as its potassium, sodium and triethanol amine salts (e.g. Eusolex® 232),

3,3′-(1,4-phenylenedimethylene)-bis-(7,7-dimethyl-2-oxobicyclo[2.2.1 ]hept-1 -yl methane sulphonic acid as well as its salts (e.g. Mexoryl® SX) and

2,4,6-trianilino-(p-carbo-2′-ethylhexyl-1′-oxy) 1,3,5-triazine (e.g. Uvinul® T 150).

These organic UV filters are typically incorporated into the compositions of the present invention in an amount of 0.5 to 10 wt %, preferably 1 to 8 wt %.

Further suitable organic UV filters are, for example,

2-(2H-benzotriazol-2-yl)-4-methyl-6-(2-methyl-3-( 1, 3,3,3-tetramethyl -1-(trimethylsilyloxy)disiloxanyl)propyl) phenol (e.g. Silatrizole®),

4,4′-[(6-[4-((1,1-dimethylethyl)aminocarbonyl)phenylamino] 1,3,5-triazin-2,3-diyl)diamino] bis-benzoic acid 2-ethylhexylester (e.g. Uvasorb® HEB),

α-(trimethylsilyl)-ω[trimethylsilyl)oxy]poly[oxy(dimethyl] [and approx. 6% methyl[2-[p -[2,2-bis-(ethoxycarbonyl]vinyl]phenoxy]-1-methylene ethyl] and approx. 1.5% methyl[3-[p-[2,2-bis-(ethoxycarbonyl)vinyl)phenoxy) propenyl) and 0.1 to 0.4% (methylhydrogen]silylene]] (n≈60) (e.g. Parsol® SLX),

2,2′-methylene-bis-(6-(2H-benzotriazol-2-yl) 4-(1,1,3,3-tetramethylbutyl) phenol (e.g. Tinosorb® M),

2,2′-(1,4-phenylene)-bis-1H-benzimidazole 4,6-disulphonic acid monosodium salt,

2,2′-(1,4-phenylene)-bis-1H-benzimidazole 5-disuiphonic acid monosodium salt,

2,2′-(1,4-phenylene)-bis-1H-benzimidazole 5-disulphonic acid monopotassium salt, and

2,4-bis-[4-(2-ethyl-hexyloxy)-2-hydroxyl]-phenyl 6-(4-methoxyphenyl) 1,3,5-triazine (e.g. Tinosorb® S).

These organic filters are typically incorporated into the compositions of the present. invention in an amount of 0.5 to 20 wt %, preferably 1 to 15 wt %.

Inorganic UV filters can be selected from the group of titanium dioxides, e.g. coated titanium dioxide (e.g. Eusolex® T-2000 or Eusolex® T-Aqua), zinc oxide (e.g. Sachtotec®), iron oxides or also cerium oxides. These inorganic UV filters are typically incorporated into the compositions of the present invention in an amount of 0.5 to 20 wt %, preferably 2 to 10 wt %.

Preferred UV filters are zinc oxide, titanium dioxide, 3-(4′-methylbenzylidene) dl-camphor, 1-(4-tert-butylphenyl) 3-(4-methoxyphenyl)propan-1,3-dione, 4-isopropyl dibenzoyl methane, 2-hydroxy-4-methoxybenzophenone, 4-methoxy cinnamic acid 2-ethylhexyl ester, 3,3,5-trimethylcyclohexyl salicylate, 4-(dimethylamino)benzoic acid 2-ethylhexyl ester, 2-cyano-3,3-diphenyl acrylic acid 2-ethylhexyl ester, 2-phenyl benzimidazole-5-sulphonic acid, as well as their potassium, sodium and triethanol amine salts.

Especially preferred UV filters are zinc oxide and titanium dioxide.

From the compositions of the present invention containing titanium dioxide, those are preferred which contain apart from titanium dioxide, in addition, one or more further UV filters selected from 3-(4′-methylbenzylidene) dl-camphor, 1-(4-tert-butylphenyl)-3-(4-methoxyphenyl)propan-1,3-dione, 4-isopropyl dibenzoyl methane, 2-hydroxy -4-methoxybenzophenone, p-methoxycinnamic acid 2-ethylhexyl ester, 3,3,5-trimethylcyclohexyl salicylate, 4-(dimethylamino)benzoic acid 2-ethylhexyl ester, 2-cyano-3,3-diphenyl acrylic acid 2-ethylhexyl ester, 2-phenyl benzimidazole 5-sulphonic acid, as well as their potassium, sodium and triethanol amine salts.

From these compositions, those are particularly preferred, which include apart from titanium dioxide, in addition, the UV. filters 2-hydroxy-4-methoxy benzophenone and/or methoxy cinnamic acid octyl ester.

In order to improve the protection of the skin and an immuno suppression of the skin, the combination of aryl oximes with ectoin and ectoin derivatives is particularly effective.

Depending on its use, the composition of the present invention can contain under the circumstances further auxiliaries and/or carrier agents such as carriers, preservatives, stabilisers, solvents, vitamins, colouring agents, agents to improve the odour, film-forming agents, thickening agents and moisturisers.

Forms of using the composition of the present invention include, for example, solutions, suspensions, emulsions, pastes, ointments, gels, creams, lotions, surfactant containing cleaning preparations and oils.

Ointments, pastes, creams and gels may contain the usual carriers, e.g. animal and vegetable fats, waxes, paraffins, starch, tragacanth, cellulose derivatives, polyethylene glycols, silicones, bentonites, silica, talcum and zinc oxide, xanthane gum, glycerin, carboxypolymethylene or mixtures of these compounds.

Solvents and emulsions may contain the usual carriers, such as solvents, solubilising agents and emulsifiers, e.g. water, ethanol, isopropanol, ethyl carbonate, ethyl acetate, benzyl alcohol, benzyl benzoate, propylene glycol, 1,3-butyl glycol, oils, in particular, cottonseed oil, peanut oil, maizegerm oil, olive oil, castor oil and sesame oil, esters of glycerin fatty acids, polyethylene glycols, xanthane gum, glycerin, carboxypolymethylene and fatty acid esters of sorbitan or mixtures of these compounds.

Suspensions may contain the usual carriers, such as liquid diluents, e.g. water, ethanol or propylene glycol, suspending agents, e.g. ethoxylated isostearyl alcohols, polyoxyethylene sorbitan esters and polyoxyethyl sorbitan esters, microcrystalline cellulose, aluminium metahydroxide, bentonite, agar-agar gum and tragacanth, xanthane gum, glycerin, carboxypolymethylene or mixtures of these compounds.

Surfactant containing cleaning products may contain the usual carriers such as salts of fatty alcohol sulphates, fatty alcohol ether sulphates, sulphosuccinic acid semi-esters, fatty acid protein hydrolysates, isothionates, imidazolinium derivatives, methyl taurates, sarcosinates, fatty acid amide ether sulphates, alkyl amidobetaines, fatty alcohols, fatty acid glycerides, fatty acid diethanol amides, vegetable and synthetic oils, lanolin derivatives, ethoxylated glycerin fatty acid esters or mixtures of these compounds.

Facial and body oils may contain the usual carriers such as synthetic oils, such as fatty acid esters, fatty alcohols, silicone oils, natural oils such as vegetable oils and oily plant extracts, paraffin oils, lanolin oils or mixtures of these compounds.

The composition of the present invention may be prepared according to usual processes. Different process techniques can be employed in order to mix the components of the composition according to the present invention as closely as possible. Depending on the required mixing intensity, one or more processes of blending compounds are used which may run subsequently or in a parallel order.

In chemical process engineering, one understands by the term “mixing” basic operations which lead to a homogenisation of compounds to the greatest possible extent. Compound streams should be combined in such a manner so that in partial volumes of the.formed mixture a composition of the single components which is as homogeneous as possible is obtained.

A special form of mixing is termed homogenisation. This is a mixing of phases which are normally immiscible with each other. Thus, homogenisation is, therefore, a change of the distribution and the particle size of the inner phase of emulsions and suspensions, so that a homogeneous system is formed when observed under the microscope, and the distributed phase will not be deposited or does not form a cream without the action of external forces:

Dispersion is understood as a mixing of a.compound system consisting of two or more phases in which one compound (dispersed phase) is distributed (dispersed) most finely in another one (dispersing agent). The particles of the dispersed phase as well as the dispersing agent may be solid, liquid or gaseous. Examples of dispersions are aerosols, emulsions, suspensions and colloids.

Another usual type of mixing in the preparation of cosmetics is emulsification. This is the mixing of two liquids which are immiscible or only slightly miscible with each other, whereby one of the liquids is finely distributed in the other. The outer phase is described as the continuous phase or the dispersing agent, the liquid distributed therein is described as the inner, discontinuous or dispersed phase. Cosmetic emulsions consist in most cases of an aqueous polar phase and an non-polar oil phase.

Suspending, on the other hand, is the distribution of very small but not molecular particles of a solid compound or a liquid. Just like emulsions, suspensions are often optically opaque and have the tendency to deposit under the influence of gravity.

The mixing processes mentioned above are suitable for the preparation of the composition of the present invention. It is particularly preferred that the composition of the present invention is prepared by homogenisation, dispersion or emulsification.

Components (a) and (b) are mixed prior to their incorporation into a carrier in order to prepare phase A. This serves the purpose to ensure the solubility and stability of component (a) in the topical composition of the present invention. The mixing is preferably conducted while stirring and at an increased temperature. The temperature during the mixing process is preferably 60 to 100° C., more preferably 70 to 90° C. After the preparation of phase A, said phase is incorporated into a phase B. The type of phase B, and in particular, the type of carrier contained therein, are dependent on the type of use of the topical composition of the present invention which are listed above. Thus, phase B is preferably an aqueous phase or an oil phase.

The step of incorporating phase A into phase B preferably takes place as a hot/hot process, i.e. both phases are heated separately from each other before the incorporation step. The temperature is preferable 60 to 100° C., more preferably 70 to 90° C. However, in some cases a temperature of one of the phases, preferably phase B, can be 20 to 30° C. This method is called a hot/cold process. Furthermore, it is possible that both phases can be used at a temperature of 20 to 30° C. (cold/cold process).

The other components, in particular, components (c) and (d) which are optionally contained in the topical composition of the present invention can be present in phase A and/or phase B. It is also possible that these components are added after the incorporation of phase A into phase B.

The obtained topical composition in accordance with the present invention is suitable for the prophylaxis, care and/or treatment of skin diseases and/or inflammation responses of the skin. In particular, the following skin diseases and inflammation responses of the skin are mentioned:

irritative dermatitis,

toxic dermatitis,

allergic diseases of the skin or the skin adnexa,

inflammatory diseases of the skin or the skin adnexa,

UV dermatitis, and

different forms of eczema.

The invention is further illustrated by means of the following examples.

### EXAMPLE 1

Assessment of the solubilities of the binary phase behavior

2-hydroxy-5-methyl-laurophenone oxime is incorporated in an amount of 10 wt % into one of the following emulsifiers by stirring at approx. 90° C.:

(a) polyglycerin-10-tricaprylate

(b) polyglycerin-10-trilaurate

(c) polyglycerin-2-oleate

(d) sodium lauryl lactate

(e) sodium cocoyl lactate

The obtained preliminary mixtures were incorporated in an amount of 10 wt % into de-mineralized water as the carrier. The incorporation takes place with stirring at 90° C.

After cooling at room temperature, these mixtures were examined by contact microscope. As a result, it was found that liquid-crystalline myelin structures were formed at approx. 25° C. Therefore, a successful liposomal encapsulation of the active agent could be achieved.

Example 2

Assessment of the compatibility with lipophilic co-emulsifiers

The preliminary mixtures of component (a) with (b) mentioned above were combined with co-emulsifiers which build up liquid crystalline gel networks in water. Glyceryl stearate, cetearyl alcohol and sorbitan stearate were investigated as lipophilic co-emulsifiers. Thus, the following compositions were prepared:

Mixed emulsifiers on the basis of polyglyceryl-2-oleate:

| Preparation No. | 2-1 | 2-2 | 2-3 |
|---|---|---|---|
| phase A | (wt. %) | (wt. %) | (wt. %) |
| 2-hydroxy-5-methyl-laurophenone oxime | 2.25 | 2.25 | 2.25 |
| Rylo PG 29 (polyglycerin-2-oleate) | 27.75 | 27.75 | 27.75 |
| Tegin M (glycerin stearate) | 70.00 | 0.00 | 0.00 |
| Lanette O (cetearyl alcohol) | 0.00 | 70.00 | 0.00 |
| Span 60 (sorbitan stearate) | 0.00 | 0.00 | 70.00 |
| Total | 100.0 | 100.00 | 100.00 |

Mixed emulsifiers on the basis of sodium cocoyl lactate:

| Preparation No. | 2-4 | 2-5 | 2-6 |
|---|---|---|---|
| phase A | (wt. %) | (wt. %) | (wt. %) |
| 2-hydroxy-5-methyl-laurophenone oxime | 2.25 | 2.25 | 2.25 |
| Patrionic SCL (sodium cocoyl acetate) | 27.75 | 27.75 | 27.75 |
| Tegin M (glycerin stearate) | 70.00 | 0.00 | 0.00 |
| Lanette O (cetearyl alcohol) | 0.00 | 70.00 | 0.00 |
| Span 60 (sorbitan stearate) | 0.00 | 0.00 | 70.00 |
| Total | 100.0 | 100.00 | 100.00 |

The components of phase A were mixed with each other whereby the components were heated with stirring to 90° C. and were then cooled to 25° C. The obtained mixtures were incorporated in an amount of 10 wt. % each into de-mineralized water (phase B) in order to form liquid-crystalline gel networks. By doing so, phase A was heated to 90° C. Simultaneously, phase B was heated to 80° C. Phase A was added to phase B and the obtained mixture was homogenized for 1 minute. The obtained compositions showed an improved storability in comparison to conventional compositions with 2-hydroxy-5-methyl-laurophenone oxime.

Example 3

Topical composition as O/V emulsion

Phases A and B as described in the following were combined with each other in the same manner as described in examples 1 and 2, in order to prepare a topical composition.

The following compositions were prepared:

TABLE 1

| | | 3-1 | 3-2 |
|---|---|---|---|
| Trade Name | Name | (wt. %) | (wt. %) |
| phase A | | | |
| Rylo PG 29 | polyglycerin-2-oleate | 2.00 | 1.00 |
| Tegin M | glycerin stearate | 1.00 | 1.00 |
| Span 60 | sorbitan stearate | 3.00 | 3.00 |
| 2-hydroxy-5-methyl- | | 0.50 | 0.50 |
| laurophenone oxime | | | |
| Miglyol 812 | capric/caprylic acid | 16.00 | 17.00 |
| | triglyceride | | |
| phase B | | | |
| Glycerin | | 6.00 | 6.00 |
| Keltrol | xanthane gum | 0.30 | 0.30 |
| Demin. water | | 71.20 | 71.20 |
| Total | | 100.00 | 100.00 |

TABLE 2

| | | 3-3 |
|---|---|---|
| Trade Name | Name | (wt. %) |
| phase A | | |
| Pationic SCL | Na cocoyl lactate | 1.00 |
| Tegin M | glycerin stearate | 1.00 |
| Span 60 | sorbitan stearate | 3.00 |
| 2-hydroxy-5-methyl- | | 0.50 |
| laurophenone oxime | | |
| Miglyol 812 | capric/caprylic acid | 16.00 |
| | triglyceride | |
| phase B | | |
| Glycerin | | 6.00 |
| Keltrol | xanthane gum | 0.30 |
| Demin. water | | 71.20 |
| Total | | 100.00 |

TABLE 3

| | | 3-4 | 3-5 | 3-6 | 3-7 |
|---|---|---|---|---|---|
| Trade Name | Name | (wt. %) | (wt. %) | (wt. %) | (wt. %) |
| phase A* | | | | | |
| Biobase EP | glycerin stearate, | 4.50 | 4.50 | 4.50 | 4.50 |
| | cetearyl alcohol, | | | | |
| | Na stearoyl lactate, | | | | |
| | lecithin | | | | |
| Miglyol 812 | capric/caprylic acid | 15.25 | 15.00 | 14.75 | 14.50 |
| | triglyceride | | | | |
| 2-hydroxy-5-methyl- | | 0.25 | 0.50 | 0.75 | 1.00 |
| laurophenone oxime | | | | | |
| phase B | | | | | |
| ETD 2050 | carbomer | 0.30 | 0.30 | 0.30 | 0.30 |
| Demin. Water | | 79.70 | 79.70 | 79.70 | 79.70 |
| Total | | 100.00 | 100.00 | 100.00 | 100.00 |

*Phases A and B were heated to 70° C. each and were mixed with each other at this temperature.

The obtained emulsions showed no crystallization of the active agent at room temperature, 40° C. and 5 defrosting/freezing cycles between −18° C. and −40° C. over a period of 4 weeks.

Example 4

Topical composition as a W/O emulsion

| | | | 4-1 | 4-2 | 4-3 |
|---|---|---|---|---|---|
| | Raw material | INCI name | (wt. %) | (wt. %) | (wt. %) |
| A | Isolan PDI | diisostearoyl | 3.00 | 3.00 | 3.00 |
| | | polyglyceryl-3- | | | |
| | | diisostearate | | | |
| | Parafin oil, liq. | mineral oil | 17.00 | 17.00 | 17.00 |
| | Isopropyl myristate | isopropyl myristate | 5.00 | 5.00 | 5.00 |
| | Bees wax | bees wax | 0.20 | 0.20 | 0.20 |
| | Cutina HR | hydrogenated | 0.30 | 0.30 | 0.30 |
| | | castor oil | | | |
| B | Demin. Water | aqua | to 100 | to 100 | to 100 |
| | Glycerin (87%) | glycerin | 4.00 | 4.00 | 4.00 |
| | Magnesium sulfate | magnesium sulfate | 1.00 | 1.00 | 1.00 |
| | Germaben II-E | propylene glycol, | 10.00 | 10.00 | 10.00 |
| | | diazolidinyl urea, | | | |
| | | methylparaben, | | | |
| | | propylparaben urea | | | |
| C | 2-hydroxy-5-methyl- | 2-hydroxy-5-methyl- | 1.00 | 2.00 | 4.00 |
| | laurophenone oxime | laurophenone oxime | | | |

Phases A and B were heated to 75° C. B was added to A with stirring. Afterwards, the mixture was homogenized at 9000 rpm for 2 minutes with the Turrax. The obtained mixture was cooled to between 30 and 35° C. and C was stirred in.

Example 5

Topical composition as a W/O emulsion

| | | | 5-1 | 5-2 | 5-3 |
|---|---|---|---|---|---|
| | Raw material | INCI name | (wt. %) | (wt. %) | (wt. %) |
| A | Arlacel 1689 | | 6.00 | 6.00 | 6.00 |
| | Parafin oil, liq. | mineral oil | 10.00 | 10.00 | 10.00 |
| | Miglyol 812 | caprylic/capric | 5.00 | 5.00 | 5.00 |
| | | triglyceride | | | |
| B | Demin. Water | aqua | to 100 | to 100 | to 100 |
| | Glycerin (87%) | glycerin | 4.00 | 4.00 | 4.00 |
| | Magnesium sulfate | magnesium sulfate | 0.50 | 0.50 | 0.50 |
| | Germaben II-E | propylene glycol, | 0.50 | 0.50 | 0.50 |
| | | diazolidinyl urea, | | | |
| | | methylparaben, | | | |
| | | propylparaben urea | | | |
| | Urea | urea | 10.00 | 10.00 | 10.00 |
| C | 2-hydroxy-5-methyl- | 2-hydroxy-5-methyl- | 1.00 | 2.00 | 4.00 |
| | laurophenone oxime | laurophenone oxime | | | |

Phases A and B were heated to 75° C. B was added to A with stirring. Afterwards, the mixture was homogenized at 9000 rpm for 2 minutes with the Turrax. The obtained mixture was cooled to between 30 and 35° C. and C was stirred in.

Example 6

Topical composition as a W/O emulsion

| | | | 5-1 | 5-2 | 5-3 |
|---|---|---|---|---|---|
| | Raw material | INCI name | (wt. %) | (wt. %) | (wt. %) |
| A | Paraffin, viscous | mineral oil | 36.9 | 36.9 | 36.9 |
| | Miglyol 812 | caprylic/capric | 10 | 10 | 10 |
| | | triglyceride | | | |
| | Wax, bleached | cera alba | 4 | 4 | 4 |
| | (1.11544) | | | | |
| | Cutina CP | cetyl palmitate | 3 | 3 | 3 |
| B | Demin. Water | aqua | to 100 | to 100 | to 100 |
| | Propylene glycol | Propylene glycol | 10 | 10 | 10 |
| | (1.07478) | | | | |
| | Germaben II-E | propylene glycol, | 0.5 | 0.5 | 0.5 |
| | | diazolidinyl urea, | | | |
| | | methylparaben, | | | |
| | | propylparaben | | | |
| | Urea | urea | 10 | 10 | 10 |
| | (108486) | | | | |
| C | 2-hydroxy-5-methyl- | 2-hydroxy-5-methyl- | 1 | 2 | 4 |
| | laurophenone oxime | laurophenone oxime | | | |

The oil phase (A) and the aqueous phase (B) were separately heated to 75° C. in a water steam bath. Phase B was slowly added to A with stirring (propeller agitator, 500 rpm, duration of addition of approx. 1 min.). Afterwards, the emulsion was homogenized (Ultra Turax, 9000 rpm, 2 min.). The obtained mixture was cooled to 35° C. with stirring and phase C was added. The stirring rate was adjusted so that the emulsion was continuously in a homogeneous movement and no air was stirred in, e.g. with a propeller agitator, 500 rpm.

Example 7

Examination of the influence of UV light induced erythema by topical pre- and post-treatment with a formulation containing an active agent in comparison to a placebo.

The objects of the present examination are the visual assessment for the determination of the influence of UV induced erythema of differing intensity by a topical formulation containing an active agent in comparison to a placebo. The formulation was employed both before and after erythema induction.

The examination was conducted as a double-blind study. Thirteen volunteers participated in the examination, all of which completed the examination correctly and completely. Four examination areas of 37 cm² each on the lower back half were defined per volunteer whereby two examination areas were pre-treated prior to the erythema induction with examination preparations in quantities of 2.0 mg/cm² each. On two further examination areas the treatment with the examination preparations was effected with the same amounts but after the erythema induction.

The examination areas of each volunteer were summarized, thus, as follows:

Examination area 1: Pre-treatment UV-exposure, examination preparation placebo,

Examination area 2: Pre-treatment UV-exposure, examination preparation active agent,

Examination area 3: Post-treatment UV-exposure, examination preparation placebo,

Examination area 4: Post-treatment UV-exposure, examination preparation active agent.

The examination preparations were composed of the following ingredients and were formulated as O/W emulsions.

| | | | wt. % | |
|---|---|---|---|---|
| | | | Examination | Examination |
| | | | preparation | preparation |
| Raw material | Batch | INCI name | active agent | placebo |
| Biobase EP | 393/97 | glycerin stearate, | 4.50 | 4.50 |
| | | cetearyl alcohol, | | |
| | | sodium stearoyl | | |
| | | lactate, lecithin | | |
| Miglyol 812 | K26777507 | caprylic/capric | 15.00 | 16.00 |
| | | triglyceride | | |
| 2-hydroxy-5- | 99/FA/024 | (2-hydroxy-5-methyl- | 1.00 | — |
| methyl- | | laurophenone | | |
| laurophenone | | oxime) | | |
| oxime | | | | |
| ETD 2050 | C418003 | carbomer | 0.30 | 0.30 |
| Demin. water | | aqua | to 100 | to 100 |
| Glycerin (87%) | K26164291907 | glycerin | 3.00 | 3.00 |
| Germaben II-E | GBTE-204 | propylene glycol | 0.50 | 0.50 |
| | | (and) diazolidinyl | | |
| | | urea | | |
| NaOH (10%) | 70164760 | sodium hydroxide | 0.60 | 0.50 |

The examination areas 1 and 2 were treated in total five times prior to the erythema induction with the respective examination preparation. This pre-treatment was conducted on the first and second day prior to the erythema induction, twice daily as well as one hour before the erythema induction.

The post-treatment of the respective examination areas 3 and 4 was conducted in total four times and started immediately after UV exposure. It was repeated on the day of the erythema induction once and on the following day twice.

For the induction of UV erythema, the examination areas were irradiated by means of a sun-simulator (SOL 500, Dr. Hönle) with the following doses: 0; 0.5, 1.0, 1.25, 1.50 and 1.75 MED (minimum erythema dose). The determination of the MED of each volunteer was conducted two days prior to the UV exposure of the examination areas on a separate area on the back. Thus, the irradiation times corresponded to the obtained individual MED, so that an irradiation with the doses mentioned above was ensured.

Twenty-four hours after the erythema induction on the individual examination areas of the thirteen volunteers, the visual assessment of the erythemas according to an assessment scale was effected:

| Visable skin change | Assessment |
|---|---|
| no erythema | 0 |
| indistinct erythema | 0.5 |
| weak erythema | 1/1.5 |
| medium erythema | 2/2.5 |
| strong erythema | 3/3.5 |
| strong erythema with vesicles | 4 |

From the obtained assessment of the volunteers mean values were calculated. These mean values of the visual assessment of the erythema are shown graphically in FIGS. 1 and 2 and are summarized in the following Table.

| | Examination | Examination | Examination | Examination |
|---|---|---|---|---|
| MED | area 1 | area 2 | area 3 | area 4 |
| 0 | 0 | 0 | 0 | 0 |
| 0.5 | 0.25 | 0.13 | 0.29 | 0.21 |
| 1.0 | 1.13 | 0.79 | 1.29 | 1.13 |
| 1.25 | 1.5 | 1.16 | 1.75 | 1.5 |
| 1.5 | 1.96 | 1.58 | 2.21 | 2.04 |
| 1.75 | 2.5 | 2.13 | 2.63 | 2.42 |

Taking into account the visual assessment, statistically significant differences between the treatment with the active agent and the placebo for the assessment of UV light induced erythema occured, whereby the treatment with the active agent reduced the degree of the erythemas, both during the pre-treatment and the post-treatment. The noted differences relate to a difference of 0.5 assessment points of the visual assessment scale.

The conductive mexameter measurements also resulted in a significant positive effect of the active agent both for the pre-treatment and also the post-treatment of UV light induced erythemas (not shown).

1. A topical composition, comprising
(a) at least one aryl oxime of the Formula (I) and
(b) at least one emulsifier, 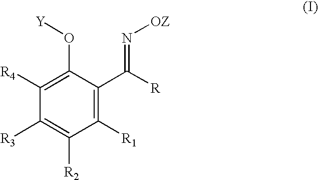
wherein:
Y,Z represent independently from each other H, C₁₋₁₈ alkyl, C₂₋₁₈ alkenyl, C₂₋₁₈ carboxy alkyl, C₃₋₁₈ carboxy alkenyl or C₂₋₁₈ alkanoyl;
R represents C₁₋₁₈ alkyl, C₂₋₁₈ alkenyl, C₃₋₈ cycloalkyl, aryl, aralkyl, heteroaryl, heteroaralkyl or condensed systems,
R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl,
wherein the component (b) is selected from the group consisting of an ester, the carboxylic acid residue of which is derived from C₅-C₁₆ acids and the hydroxyl residue of which is derived from monomers, dimers or trimers of lactic acid or one of its salts or a polyglycerin of 2 to 10 molecules of glycerin whereby 1 to 3 moles of carboxylic acid are present per mole of polyglycenin.

2. Topical composition according to claim 1, wherein the component (a) is present in the composition in an amount of 0.02 to 2 wt. %

3. Topical composition according to claim 1 or 2, wherein the component (b) is selected from the group consisting of polyglycerin 10-tricaprylate, polyglycerin -10-trilaurate, polyglycerin-2-oleate, sodium lauroyl lactate, sodium cocoyl lactate, capric/caprylic acid triglyceride and mixtures thereof.

4. Topical composition according to one of claims 1 to 3 wherein the component (b) is present in the composition in an amount of 0.5 to 30 wt. %.

5. Topical composition according to one of claims 1 to 4, wherein the composition further comprises (c) at least one co-emulsifier selected from the group consisting of glycerin and sorbitan ester derivatives, cetearyl alcohol, cetearyl ester derivatives and mixtures thereof.

6. Topical composition according to claim 5, wherein component (c) is present in the composition in an amount of 0.1 to 40 wt. %.

7. Topical composition according to one of claims 1 to 6, wherein the composition further comprises (d) at least one lipophilic solvent.

8. Topical composition according to claim 7, wherein the component (d) is selected from the group consisting of ethyl butyl acetyl amino propionate, ethanol, isopropanol, isopropyl myristate and mixtures thereof.

9. Topical composition according to claims 7 or 8, wherein the component (d) is present in the composition in an amount of 0.1 to 20 wt. %.

10. Topical composition according to one of claims 1 to 9, wherein the composition further comprises (e) at least one auxiliary, selected from anti-oxidants and UV filters.

11. Process for the preparation of a topical composition according to one of claims 1 to 10, comprising the following steps
preparation of a phase A by mixing the components (a) and (b), and
incorporation of the obtained phase A into a phase B, containing a carrier.

12. Process according to claim 11, wherein the carrier comprises de-ionized water, as well as mixtures of de-ionized water and alcohols.

13. Process according to claims 11 or 12, wherein phase A and/or phase B further contain at least one of the components (c) to (d).

14. Process according to one of claims 11 to 13, wherein phase A is prepared at 60 to 100° C. with stirring.

15. Process according to one of claims 11 to 14, wherein the phases A and/or B are heated to 60 to 100° C. prior to the incorporation step.

16. Use of the topical composition according to one of claims 1 to 10 for the prophylaxis and/or treatment of skin diseases and/or inflammation responses of the skin.

17. Use of the topical composition according to one of claims 1 to 10 for the cosmetical care of the skin.
